(19) United States
(12) Patent Application Publication
(10) Pub. No.: US 2009/0120438 A1
(43) Pub. Date: May 14, 2009

(54) Breathing Assistance Device Comprising an Independent Secondary Unit

(76) Inventor: Philippe Chalvignac, New South Wales (AU)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 11/922,389

(22) PCT Filed: Jun. 23, 2005
(86) PCT No.: PCT/IB2005/002326

§ 371 Date: Dec. 24, 2008

(51) Int. Cl. A61M 16/12

(52) U.S. Cl. 128/204.21

(57) ABSTRACT

The invention relates to a breathing assistance device (1) comprising: —a first gas inlet (5) for receiving a primary respiratory gas and a first regulating means for regulating said primary respiratory gas, both being located in a central unit (2); —a second gas inlet (6) for receiving a secondary respiratory gas and a second regulating means (7) for regulating said secondary respiratory gas; —a controlling means for controlling said first and second regulating means in order to mix said primary and secondary respiratory gases in controlled respective proportions into a controlled mix of gases; —a feeding means (4) for feeding a patient with said controlled mix of gases, characterised in that said second gas inlet (5) and second regulating means (7) are both located in a secondary unit (8) removably connected to the central unit (2).

### FIELD OF THE INVENTION

The present invention concerns a breathing assistance device for a patient.

More precisely, the invention concerns a breathing assistance device comprising:
a first gas inlet for receiving a primary respiratory gas and a first regulating means for regulating said primary respiratory gas,
a second gas inlet for receiving a secondary respiratory gas and a second regulating means for regulating said secondary respiratory gas,
a controlling means for controlling said first and second regulating means in order to mix said primary and secondary respiratory gases in controlled respective proportions into a controlled mix of gases,
a feeding means for feeding said patient with said controlled mix of gases.

### TECHNICAL BACKGROUND

Breathing assistance devices such as mentioned above already exist.

Such devices comprise a central unit which includes a pressurised primary gas source such as a turbine for compressing the air from the ambient atmosphere (air being thus the primary respiratory gas). The first regulating means of the breathing assistance device can correspond to the turbine itself, with its associated control means (e.g. control of the speed of rotation of the turbine).

In other devices of the art, the first regulating means comprises the turbine and additional means (such as a proportional valve).

These devices also comprise a feeding means in the form of a respiratory duct for bringing the pressurised gas to the patient.

It is furthermore possible that the device additionally comprises a secondary respiratory duct between the central unit and the patient, said second duct being dedicated to evacuating the gases expired by the patient, such as carbon dioxide.

In such devices, the inlet of the turbine corresponds to the first gas inlet for the primary respiratory gas to be provided to the patient. Alternatively, the first gas inlet can be replaced by means allowing a connection to an external pressurised primary gas source (such as a fixed source of pressurised air e.g. in a hospital).

It is further known to incorporate into such breathing assistance devices a second gas inlet for receiving a secondary respiratory gas to be provided to said patient.

The secondary respiratory gas is typically oxygen. This secondary respiratory gas is mixed to air in a controlled proportion. Therefore the breathing assistance device is provided with a second regulating means controlled by a controlling means. This allows to feed the patient with pressurised gas comprising air having been enriched with oxygen in a controlled manner.

The secondary respiratory gas is generally provided by an external secondary gas source, such as a pressurised oxygen bottle or, e.g. in hospitals, a wall outlet for providing pressurised oxygen. The second gas inlet of the known devices therefore includes a connection means for connecting the external secondary gas source to the breathing assistance device. This connection means is fixed on a wall of the central unit.

In practice, the connection means for connecting the external secondary gas source to the breathing assistance device has in itself a significant volume.

Indeed, the secondary gas is typically pressurised oxygen which is provided to the breathing assistance device at a pressure which is between 4 and 6 atmospheres.

And for safety reasons, the connection means to such a secondary gas source has to be robust enough to address the risk of leakage. The resulting connection means is thus of a significant size.

For oxygen as a secondary respiratory gas, there are standard connection means which make it possible to connect the respiratory unit to a pressurised secondary gas source wherever it is to be found. The existing devices thus use such standard connection means located on a wall of the central unit.

Finally, it is specified that the devices which include a second gas inlet for receiving a secondary respiratory gas such as oxygen are in practice used either for feeding the patient with a mix of primary and secondary respiratory gases, or with primary respiratory gas only. In other words, in many cases enriching the primary respiratory gas with the secondary respiratory gas is an option which is not permanently used.

To this end, a mixing means (such as a three way valve) is generally provided within the central unit to mix, only when it is desired, said primary and secondary respiratory gases.

The above being exposed, it is furthermore to be specified that a current trend of design for breathing assistance devices is to try to make them as small as possible.

It is indeed desired to obtain a device as small as possible, this increasing in particular the ability for the patient to move freely while being connected to the breathing assistance device.

However, if it is desired to be able to connect a secondary respiratory gas, the large size of the connecting means mentioned above is a limitation to the miniaturization of the breathing assistance device, and more precisely to its central unit.

The second regulating means constitutes a further limitation to miniaturization.

It is thus an object of the invention to overcome such limitation.

### SUMMARY OF THE INVENTION

In order to attain the above-mentioned object, the invention proposes a breathing assistance device as defined in claim 1.

In particular, the invention concerns a breathing assistance device for a patient comprising:
a first gas inlet for receiving a primary respiratory gas and a first regulating means for regulating said primary respiratory gas, said first gas inlet and first regulating means being both located in a central unit of the breathing assistance device;
a second gas inlet for receiving a secondary respiratory gas and a second regulating means for regulating said secondary respiratory gas;
a controlling means for controlling said first and second regulating means in order to mix said primary and secondary respiratory gases in controlled respective proportions into a controlled mix of gases;
a feeding means for feeding said patient with said controlled mix of gases, said feeding means being located between the central unit and the patient;
   characterised in that said second gas inlet and second regulating means are both located in a secondary unit of the breathing assistance device, said secondary unit being removably connected to the central unit.

Preferable but not limited aspects of such a breathing assistance device are the following:
the secondary unit is removably connected to the central unit via a male and female joint or by a connection duct for the secondary respiratory gas, the connection duct having a diameter from 4 to 8 mm;
the second gas inlet comprises a connection means being adapted to be connected to an external pressurised gas source;
the second regulating means comprises a pressure reducing means and/or a proportional means that may be electrically operated;
the controlling means is located within the central unit;
the second regulating means is controlled by the controlling means;
the secondary unit comprises a data link allowing a transmission of data between the second regulating means and the controlling means of the central unit;
the secondary unit comprises a power supply link for transmitting power from the central unit to the second regulating means;
the secondary unit comprises a controlling electronic board being interposed between the regulating means and the data and power supply links, and being adapted to process data for controlling the second regulating means;
the secondary unit comprises at least a gas flow sensor and a pressure sensor, both being connected to the controlling electronic board;
the secondary unit comprises a central groove for storing said connection duct, so that the secondary unit may have a diabolo shape.

According to a further aspect of the invention, there is proposed a secondary unit for a breathing assistance device, the breathing assistance device being capable of feeding a patient with a mix of at least two respiratory gases and comprising a central unit, characterised in that the secondary unit is removably connected to the central unit, and is adapted to feed the central unit with a regulated flow of a respiratory gas coming from an external pressurised gas source.

Preferable but not limited aspects of such a secondary unit are the following:
the secondary unit is removably connected to the central unit via a male and female joint or by a connection duct, the connection duct having a diameter from 4 to 8 mm;
the secondary unit comprises a single inlet having a connection means adapted to be connected to an external pressurised gas source;
the secondary unit comprises a power supply link for transmitting power from the central unit to secondary unit;
the secondary unit comprises a single inlet having a connection means adapted to be connected to an external pressurised gas source;
the secondary unit comprises a regulating means for regulating said respiratory gas, the regulating means comprising a pressure reducing means and/or a proportional means;
the secondary unit comprises a controlling electronic card for controlling the regulating means, the data and power supply links being connected to the controlling electronic card;
the secondary unit comprises a central groove for storing the connection duct, so that the secondary unit may have a diabolo shape.

According to another aspect of the invention, there is provided a central unit for a breathing assistance device, the breathing assistance device being capable of feeding a patient with a mix of gases comprising a primary respiratory gas and a secondary respiratory gas, characterised in that the central unit comprises a gas inlet for receiving a regulated flow of the secondary respiratory gas, and a mixing means for selectively mixing said regulated flow of the secondary gas with said primary gas in order to obtain the desired mix of gases.

Preferable but not limited aspects of such a central unit are the following:
the mixing means is a three way valve;
the central unit comprises a controlling means for controlling a regulating means located outside said central unit, said regulating means regulating the flow of the secondary respiratory gas that reaches the inlet of the central unit;
the inlet is adapted for receiving ducts having diameters from 4 to 8 mm.

### BRIEF DESCRIPTION OF THE DRAWINGS

Other characteristics and advantages of the invention will become clear from the following description which is only given for illustrative purposes and is in no way limitative and should be read with reference to the attached drawings on which:

FIG. 1 is a schematic representation of a breathing assistance device according to the invention;

FIG. 2 is a schematic representation of a breathing assistance device according to another embodiment of the invention;

FIG. 3 is a schematic representation of a secondary unit of a breathing assistance device according to the invention;

FIG. 4 is a schematic sectional view of an embodiment of a secondary unit of the breathing assistance device according to the invention.

### DETAILED DESCRIPTION OF THE DRAWINGS

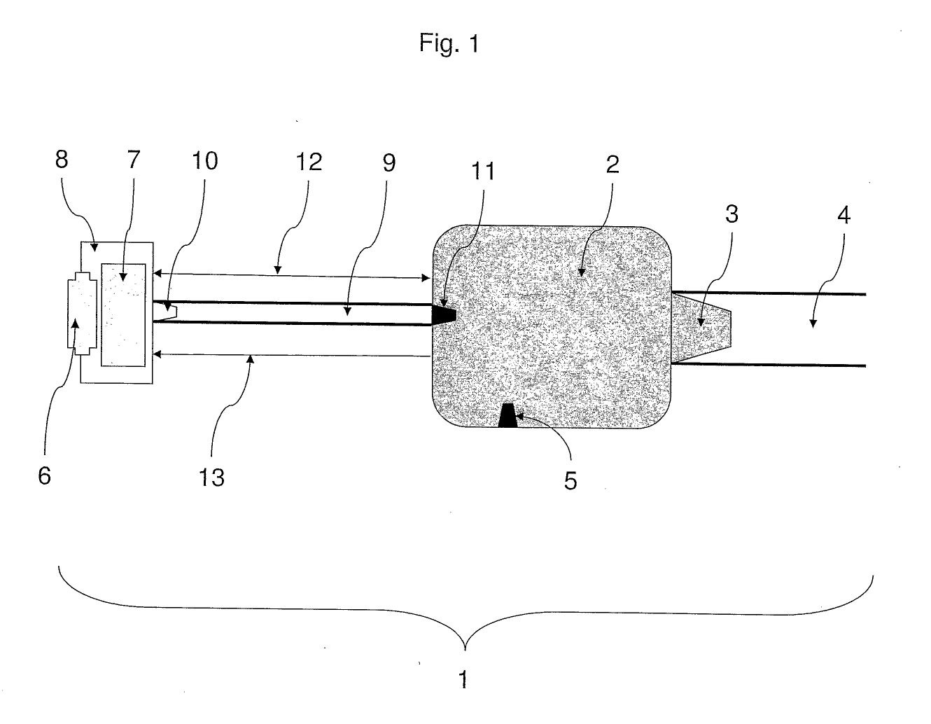

We shall first describe the breathing assistance device of the invention with reference to FIG. 1.

The breathing assistance device 1 of the invention comprises a central unit 2 that is capable of providing a respiratory gas to a patient.

The central unit 2 is provided with an outlet 3 from which the respiratory gas is brought from the central unit 2 to the patient via a feeding means 4. Such an outlet 3 may be for instance a rubber element having a tapered shape.

The feeding means 4 is connected to the outlet 3 of the central unit 2 and allows the patient to be fed with the respiratory gas coming from the central unit 2 of the breathing assistance device 1.

This feeding means 4 may constitute the single respiratory duct between the patient and the central unit 2.

The proximal end of this duct is generally connected to the patient with a device adapted for tracheotomy or a mask.

Alternatively, the feeding means 4 may be associated to a second duct for the patient to reject expiratory gases.

The breathing assistance device 1 of the invention is foreseen for providing the patient with a respiratory gas being composed of a mix of a primary respiratory gas and a secondary respiratory gas, both gases being provided in controlled proportions.

Therefore the central unit 2 of the breathing assistance device 1 of the invention comprises a first gas inlet 5 for receiving a primary respiratory gas and a first regulating means (not represented) for regulating said primary respiratory gas.

The first gas inlet 5 is arranged for collecting ambient air, the ambient air being the primary respiratory gas to be provided to the patient.

Alternatively, the first gas inlet can be replaced by means allowing a connection to an external pressurised primary gas source (such as a fixed source of pressurised air, e.g. in a hospital).

The first regulating means is generally a turbine, preferably of a small size, which provides a controlled flow of the primary respiratory gas. This turbine may optionally be associated to another regulating device, such as a proportional valve, in order to increase the control of the flow of the primary respiratory gas.

The breathing assistance device 1 of the invention is also capable of providing the patient with a secondary respiratory gas.

To this end, the breathing assistance device 1 of the invention comprises a second gas inlet 6 and a second regulating means 7 for respectively receiving and controlling a secondary respiratory gas.

As exposed above, the second gas inlet 6 has a significant size (due in particular to security standards).

However, according to the invention, the second gas inlet 6 and the second regulating means 7 are not located within the central unit 2. They are namely located in a secondary unit 8 which is distinct from the central unit 2.

A mixing means, such as a three way valve, may be provided within the central unit 2 to mix said primary and secondary respiratory gases.

Using such a secondary unit 8 transforms the breathing assistance device in a modular breathing assistance device.

The secondary unit 8 is namely removably connected to the central unit 2. Therefore when it is desired to feed the patient with a mix of respiratory gases, that is comprising a secondary respiratory gas, the secondary unit 8 (connected to an external respiratory gas source) is coupled with the central unit 2.

When the patient needs to be fed only with the primary respiratory gas, the secondary unit 8 is not used. In this case, the breathing assistance device 1 consists only in the central unit 2.

When the secondary unit 8 and the central unit 2 are coupled, a connection duct 9 may thus be provided between the secondary unit 8 and the central unit 2.

Such a connection duct 9 allows a transmission of the secondary respiratory gas from the secondary unit 8 to the central unit 2. The connection duct 9 is namely connected to a gas outlet 10 (such as a rubber element having a tapered shape) of the secondary unit 8 at one end and to a gas inlet 11 of the central unit 2 at the other end.

As the secondary respiratory gas is regulated by the second regulating means 7 within the secondary unit 8, e.g. regarding its pressure, the security standards that the connection duct 9 has to fulfil are reduced. Therefore, the connection duct 9 may have dimensions significantly less critical than the dimensions of standard ducts (that usually connect the external respiratory gas source to the breathing assistance device). Indeed, the connection duct 9 may have a diameter between 4 and 8 mm (that is relatively smaller than the diameter of standard ducts comprised between 12 and 16 mm) and is relatively flexible, thin and light.

Moreover the gas inlet 11 of the central unit 2 is adapted to be connected with the connection duct 9 and is therefore of small dimensions. The gas inlet 11 of the central unit 2 is namely particularly small relative to the second gas inlet 6 of the secondary unit 8.

Having such a small gas inlet 11 in the central unit 2 is a great asset, especially when the breathing assistance device 1 is to be used only for a primary respiratory gas. Indeed in this case, as exposed above, the breathing assistance device 1 consists only in the central unit 2 and is consequently small and light.

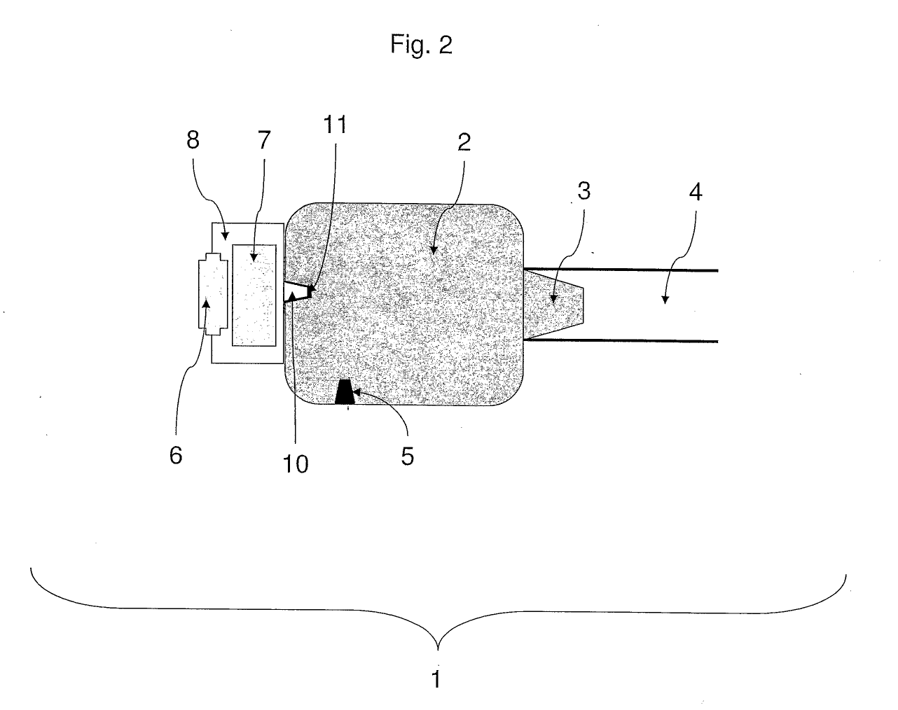

According to another embodiment of the invention as illustrated in FIG. 2, the secondary unit 8 is manufactured such that it may be directly coupled to the central unit 2, that is without any connection duct.

The secondary unit 8 may for example have a shape to be directly plugged on one side of the central unit 2. In this example, the central unit 2 and the secondary unit 8 may be coupled via a male and female joint.

In this case, the gas outlet 10 of the secondary unit 8 is adapted to be directly connected with the gas inlet 11 of the central unit 2. Once again, both gas outlet 10 and gas inlet 11 may be of small dimensions. Indeed, the secondary respiratory gas having been regulated within the secondary unit 8, e.g. regarding its pressure, the security standards that the gas outlet 10, and consequently the gas inlet 11, have to fulfil are reduced.

The breathing assistance device 1 of the invention further comprises a data link 12 provided between the secondary unit 8 and the central unit 2.

Indeed, the central unit 2 comprises a controlling means (not represented) for controlling, among others, the second regulating means 7 of the secondary unit 8.

This control is made possible thanks to the data link 12 which connects the controlling means of the central unit 2 and the second regulating means 7.

In a preferred embodiment of the invention, a power supply link 13 may be provided between the secondary unit 8 and the central unit 2.

Indeed, the regulating means 7 may be electrically operated and will in this case need a power supply. The central unit 2 being generally provided with a power supply, such as an internal battery or an external power supply, the power supply link 13 connected to the power supply of the central unit 2 feeds the second regulating means 7 with power when needed.

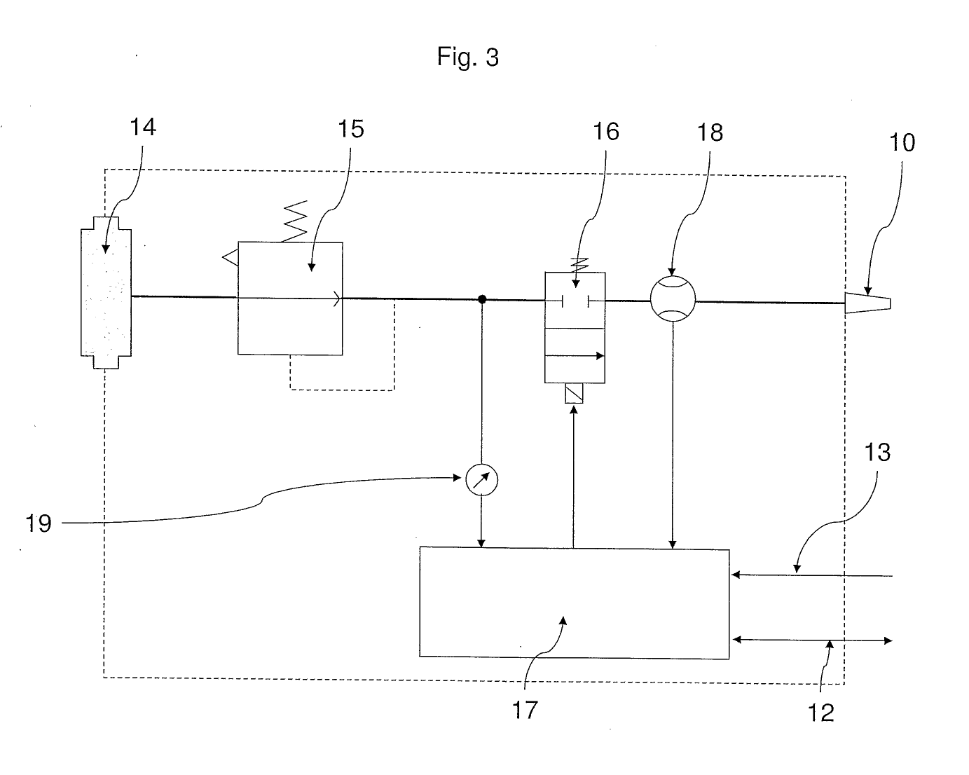

FIG. 3 is a schematic representation of a secondary unit 8 of the breathing assistance device 1 according to the invention.

This secondary unit 8 comprises a connection means 14 which, corresponds to the second gas inlet 6.

The connection means 14 is adapted to be connected to an external pressurised source which provides a secondary respiratory gas.

As stated above, the secondary respiratory gas is typically pressurised oxygen which is provided to the breathing assistance device 1 at a pressure between 4 and 6 atmospheres.

Therefore, the connection means 14 has to be robust enough to address a risk of leakage, and is thus of a significant size.

As a consequence, the breathing assistance device 1 of the invention is preferably provided with a secondary unit 8 having a standard connection means 14, which make it possible to connect the breathing assistance device 1 to a pressurised secondary gas source wherever it is to be found.

As described before, the secondary unit 8 comprises a second gas regulating means 7.

The object of this second gas regulating means 7 is to control the secondary respiratory gas to be provided from the secondary unit 8 to the central unit 2 through the connection duct 9.

Indeed, the external pressurised gas source generally provides a secondary respiratory gas at high pressures. As the secondary respiratory gas is to be provided to the patient, this pressure has to be decreased. Therefore the second regulating means 7 comprises a pressure reducing means 15, such as a reducing valve.

The second regulating means 7 may further comprise a proportional means 16, such as a proportional valve. This proportional means 16 is capable of controlling the flow of the secondary respiratory gas to be provided to the patient.

Preferably, the inlet of the pressure reducing means 15 is coupled with the second gas inlet 6, the outlet of the pressure reducing means 15 is coupled with the inlet of the proportional means 16, and the outlet of the proportional means is coupled with the gas outlet 10 of the secondary unit 8.

In a preferred embodiment of the invention, the proportional means 16 is electrically operated. The proportional means may therefore be a proportional electrovalve. In such a case, the proportional means 16 is powered via the power supply link 13.

The operation of the second regulating means 7, and in particular of the proportional valve 16, is made possible thanks to the data transmitted from the controlling means of the central unit 2 through the data link 12.

It is therefore possible to set the characteristics that the flow of the secondary respiratory gas has to fulfil, e.g. the pressure of oxygen, directly on the central unit 2. A data processing means is namely provided within the central unit 2 in order to command the controlling means, which then controls the second regulating means 7 of the secondary unit 8.

Such data from the controlling means may directly control the second regulating means 7, being thus directly connected to the proportional means 16 for example.

Alternatively, a controlling electronic board 17 may be provided within the secondary unit 8 for processing the data coming from the controlling means of the central unit 2, and controlling the second regulating means 7 in consequence.

The power supply link 13 is preferably connected to the controlling electronic board 17 which transmits to the second regulating means 7 the power needed.

Measurement means may further be provided within the secondary unit 8 in order to increase the control of the second regulating means 7.

Thus, there is preferably provided a gas flow sensor 18, such as a hot wire sensor, and a pressure sensor 19.

In the case the second regulating means 7 comprises both a pressure reducing means 15 and a proportional means 16, the pressure sensor 19 is arranged to measure the pressure of the secondary respiratory gas between the pressure reducing means 15 and the proportional means 16. The gas flow sensor 18 is in this case arranged to measure the flow of the secondary respiratory gas coming from the proportional means 16.

The measurement means of the secondary unit 8 are either directly connected to the controlling means of the central unit 2, or connected to this controlling means via the controlling electronic board 17 of the secondary unit 8.

Using a controlling electronic board 17 is therefore a great advantage for the miniaturisation of the device. Indeed, it allows to reduce the number of data wires between the secondary unit 8 and the central unit 2, a single data wire being necessary as the data are directly processed within the controlling electronic board 17.

Thus, the coupling of the central unit 2 and the secondary unit 8 is simple as only three links are required, that is a link for the secondary respiratory gas (directly by connecting the gas outlet 10 and the gas inlet 11 together or via a connection duct 9), a single data wire 12 and a single power supply wire 13.

Moreover, both data and power supply wires may be gathered into a single and relatively small transmission cable.

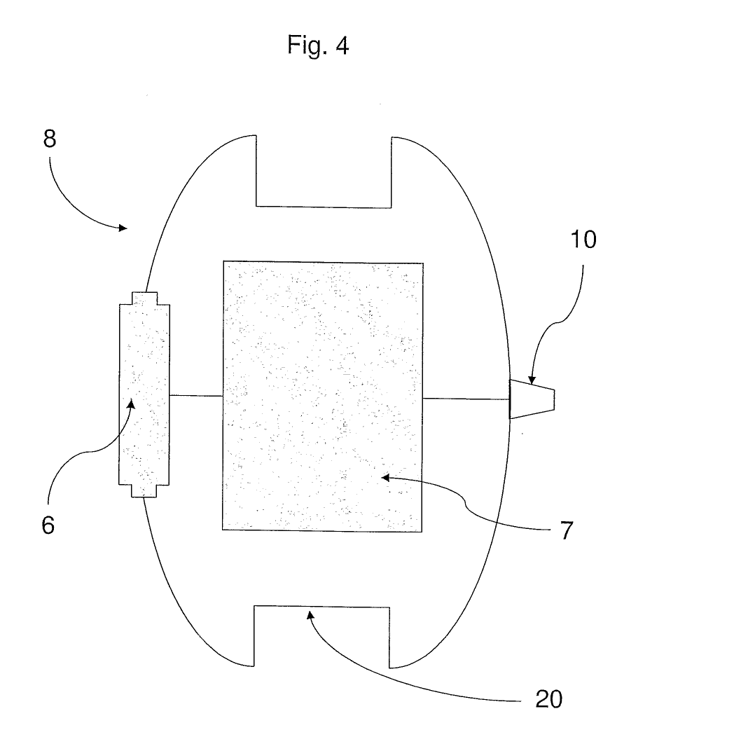

Finally, FIG. 4 is a schematic sectional view of an embodiment of the secondary unit 8 of the breathing assistance device according to the invention. Such a secondary unit 8 is to be coupled to the central unit 2 via a connection duct 9.

In this embodiment, a central groove 20 extending all around the secondary unit 8 is provided. Such a central groove 20 has dimensions so that the connection duct 9 may be stored therein. More precisely, the connection duct 9 is arranged within the groove 20 in order to be wrapped around of the secondary unit 8.

In the same manner, the data and power supply wires may be stored within the central groove 20.

Therefore, when a secondary respiratory gas has not to be provided to the patient, the secondary unit 8 may be removed from the central unit 2. to this end, the connection duct 9 and the data and power supply links (12;13) are disconnected from the central unit 2.

Such a disconnected secondary unit 8 having to be stored, it is convenient to dispose the connection duct 9, and eventually the data and power supply links (12;13), around the secondary unit 8 within the central groove 20.

A secondary unit 8 comprising a central groove 20 has for example a diabolo shape, as illustrated in FIG. 4.

1. Breathing assistance device for a patient, comprising:
a first gas inlet (5) for receiving a primary respiratory gas and a first regulating means for regulating said primary respiratory gas, said first gas inlet (5) and first regulating means being both located in a central unit (2) of the breathing assistance device;
a second gas inlet (6) for receiving a secondary respiratory gas and a second regulating means (7) for regulating said secondary respiratory gas;
a controlling means for controlling said first and second regulating means in order to mix said primary and secondary respiratory gases in controlled respective proportions into a controlled mix of gases;
a feeding means (4) for feeding said patient with said controlled mix of gases, said feeding means being located between the central unit (2) and the patient;
characterised in that said second gas inlet (5) and second regulating means (7) are both located in a secondary unit (8) of the breathing assistance device, said secondary unit (8) being removably connected to the central unit (2).

2. Breathing assistance device according to claim 1, characterised in that the secondary unit (8) is removably connected to the central unit (2) via a male and female joint.

3. Breathing assistance device according to claim 1, characterised in that the secondary unit (8) is removably connected to the central unit (2) by a connection duct (9) for the secondary respiratory gas.

4. Breathing assistance device according any one of claim 1 to 3, characterised in that the second gas inlet (6) comprises a connection means (14) being adapted to be connected to an external pressurised gas source.

5. Breathing assistance device according to any one of claims 1 to 4, characterised in that the second regulating means (7) comprises a pressure reducing means (15).

6. Breathing assistance device according to any one of claims 1 to 5, characterised in that the second regulating means (7) comprises a proportional means (16).

7. Breathing assistance device according to claim 6, characterised in that the proportional means (16) is a proportional valve being electrically operated.

8. Breathing assistance device according to any one of claims 1 to 7, characterised in that the controlling means is located within the central unit (2).

9. Breathing assistance device according to any one of claim 1 to 8, characterised in that the second regulating means (7) is controlled by the controlling means.

10. Breathing assistance device according to claim 9, characterised in that the secondary unit (8) comprises a data link (12) allowing a transmission of data between the second regulating means (7) and the controlling means of the central unit (2).

11. Breathing assistance device according to claim 10, characterised in that the secondary unit (8) comprises a power supply link (13) for transmitting power from the central unit (2) to the second regulating means (7).

12. Breathing assistance device according to claim 11, characterised in that the secondary unit (8) comprises a controlling electronic board (17) being interposed between the regulating means (7) and the data and power supply links (12,13), and being adapted to process data for controlling the second regulating means (7).

13. Breathing assistance device according to claim 12, characterised in that the secondary unit (8) comprises at least a gas flow sensor (18) and a pressure sensor (19), both being connected to the controlling electronic board (17).

14. Breathing assistance device according any one of claims 3 to 13, characterised in that the connection duct (9) between the secondary unit (8) and the central unit (2) has a diameter of 4 to 8 mm.

15. Breathing assistance device according any one of claims 3 to 14, characterised in that the secondary unit (8) comprises a central groove (20) for storing said connection duct (9).

16. Breathing assistance device according to claim 15, characterised in that the secondary unit (8) has a diabolo shape.

17. Secondary unit for a breathing assistance device (1), the breathing assistance device being capable of feeding a patient with a mix of at least two respiratory gases and comprising a central unit, characterised in that the secondary unit is removably connected to the central unit (2), and is adapted to feed the central unit (2) with a regulated flow of a respiratory gas coming from an external pressurised gas source.

18. Secondary unit according to claim 17, characterised in that said secondary unit is removably connected to the central unit (2) via a male and female joint.

19. Secondary unit according to claim 17, characterised in that said secondary unit is removably connected to the central unit (2) by a connection duct (9).

20. Secondary unit according to any one of claim 17 to 19, characterised in that said secondary unit comprises a single inlet (6) having a connection means (14) adapted to be connected to an external pressurised gas source.

21. Secondary unit according to any one of claims 17 to 20, characterised in that said secondary unit comprises a power supply link (13) for transmitting power from the central unit (2) to secondary unit (2).

22. Secondary unit according to any one of claims 17 to 21, characterised in that said secondary unit comprises a single inlet (6) having a connection means (14) adapted to be connected to an external pressurised gas source.

23. Secondary unit according to any one of claim 17 to 22, characterised in that said secondary unit comprises a regulating means (7) for regulating said respiratory gas.

24. Secondary unit according to claim 23, characterised in that said regulating means (7) comprises a pressure reducing means (15).

25. Secondary unit according to claim 23 or 24, characterised in that said regulating means (7) comprises a proportional means (16).

26. Secondary unit according to any one of claim 23 to 25, characterised in that said secondary unit comprises a controlling electronic card (17) for controlling the regulating means (7).

27. Secondary unit according to claim 26, characterised in that the data and power supply links (12;13) are connected to the controlling electronic card (17).

28. Secondary unit according to any one of claims 19 to 27, characterised in that said secondary unit comprises a central groove (20) for storing the connection duct (9).

29. Secondary unit according to claim 28, characterised in that said secondary unit has a diabolo shape.

30. Central unit for a breathing assistance device (1), the breathing assistance device (1) being capable of feeding a patient with a mix of gases comprising a primary respiratory gas and a secondary respiratory gas, characterised in that the central unit comprises a gas inlet (11) for receiving a regulated flow of the secondary respiratory gas, and a mixing means for selectively mixing said regulated flow of the secondary gas with said primary gas in order to obtain the desired mix of gases.

31. Central unit according to claim 30, characterized in that said mixing means is a three way valve.

32. Central unit according to any one of claim 30 or 31, characterised in that the central unit comprises a controlling means for controlling a regulating means located outside said central unit, said regulating means (7) regulating the flow of the secondary respiratory gas that reaches the inlet (11) of the central unit.

33. Central unit according to any one of claims 30 to 32, characterised in that the inlet (11) is adapted for receiving ducts having diameters from 4 to 8 mm.
